(19) United States
(12) Patent Application Publication
(10) Pub. No.: US 2009/0198126 A1
(43) Pub. Date: Aug. 6, 2009

(54) IMAGING SYSTEM

(76) Inventor: Klaus Klingenbeck-Regn, Nurnberg (DE)

Correspondence Address: BRINKS HOFER GILSON & LIONE, P.O. BOX 10395, CHICAGO, IL, 60610, US

(21) Appl. No.: 12/026,080

(22) Filed: Feb. 5, 2008

(51) Int. Cl. A61B 5/05

(52) U.S. Cl. 600/426

(57) ABSTRACT

An imaging system for planning and monitoring a medical procedure is provided. The imaging system includes an image processing system and an imaging device operative to obtain a 3D data set and a 2D projection data set. The imaging device is communicatively coupled to the image processing system. The image processing system is operative to image register the 3D data set and the 2D projection data set from the imaging device.

### BACKGROUND

The present embodiments relate to an imaging system for monitoring a medical procedure.

Imaging systems use imaging devices to plan and monitor medical procedures, such as needle work and other medical operations or surgeries. Prior to the medical procedure, the imaging device obtains data relating to an area to be imaged. The data represents a three-dimensional (3D) area, so may be referred to as a 3D data set. The pre-procedure 3D data set is generally obtained when the patient's body parts in the area to be imaged are motionless and in a static position. The pre-procedure 3D data set is used to plan the medical procedure and as a reference during the medical procedure.

During the medical procedure, the imaging device monitors the procedure by obtaining image data relating to the area to be imaged at periodic times. The data represents a 3D area. To obtain the medical procedure 3D data set the medical procedure may be momentarily stopped. The momentary stoppage may be caused by a need to rotate the imaging device around the area to be imaged. For monitoring, the medical procedure 3D data set is compared to the pre-intervention 3D data set. For example, the comparison may be used to monitor the progress of a needle inside a patient's body. Comparison of the pre-procedure image data and the medical procedure image data may include overlaying the images.

The body parts in the area to be imaged may move during the medical procedure. The movement of the body parts may prevent the pre-procedure image from matching the medical intervention procedure data. For example, a deflated lung during a medical procedure does not match a fully inflated lung prior to the medical intervention. Overlaying images that do not match may prevent a physician from properly monitoring the medical procedure.

### SUMMARY

The present embodiments relate to monitoring a medical procedure using image registration of a three-dimensional (3D) data set and a two-dimensional (2D) projection data set. Image registration may include translation, deformation, and/or interpolation of different points in the data set. Translation and deformation may include adjusting one or more data points in the 2D projection data set, which are marked with a fiducial marker. The fiducial marker in the 3D data set may be used as reference point for relating, aligning, or registering different images, representations, and projections, which are obtained from different sources and/or at different times.

In a first aspect, a method for monitoring a medical procedure is provided. The method includes obtaining three-dimensional (3D) representation data and two-dimensional (2D) projection data. The 3D representation data and the 2D projection data are image registered.

In a second aspect, an imaging system for planning and monitoring a medical procedure is provided. The imaging system includes an image processing system; and an imaging device operative to obtain a 3D data set and a 2D projection data set. The imaging device is communicatively coupled to the image processing system. The image processing system is operative to image register the 3D data set and the 2D projection data set from the imaging device.

In a third aspect, a computer readable storage media has stored therein data representing instructions executable for monitoring a medical procedure. The instructions include obtaining a 3D data set and a 2D projection data set; and registering the 3D data set and the 2D projection data set.

### BRIEF DESCRIPTION OF THE DRAWINGS

FIG. 1 illustrates one embodiment of an imaging system.

FIG. 2 illustrates one embodiment of an image processing system.

FIG. 3 illustrates one embodiment of image registration.

FIG. 4 illustrates one embodiment of correction vectors used to translate and deform a two-dimensional projection.

FIG. 5 is a flowchart for one embodiment of a method for monitoring a medical intervention.

### DETAILED DESCRIPTION

The present embodiments relate to an imaging system that plans and/or monitors a medical procedure. The medical procedure may be a medical intervention into a patient's organ. Monitoring the medical intervention may be done in real-time, for example, with X-ray scanning or ultrasound. Monitoring may include relating real-time image data and a static 3D data set to each other. Artificial markers, such as fiducials or X-ray absorbent balls, may be used to relate the static 3D data set, which may be taken prior to the medical intervention, and a medical intervention image taken during the medical intervention. The fiducials may be reference points. One or more fiducials may be disposed in a stationary fashion outside the patient to serve as a reference point. A plurality of fiducials may be implanted in the affected organ before the procedure. All the fiducials are mounted in such a way that they are visible in a 3D representation generated from the 3D data set and in a 2D projection generated from image data obtained during the medical intervention.

An imaging device may be used to obtain the 3D data set prior to the intervention and used to determine the 3D coordinates of the fiducials. During the intervention, a biplane system may be used to scan the imaging region from two planes. The fiducials are visible in the 2D projection image generated from the data taken during the medical intervention. The stationary fiducial is used for aligning the scanned image and the 3D data set with one another. The 3D coordinates from 3D data set and the coordinates from the planar data set are used to correct local organ shifts by local registration of the scanned images and the 3D data set.

The coordinates of the fiducials represent a plurality of discrete points in space. Correction vectors for the registration can be established using these discrete points identifiable in both data sets. In order to align the 3D data set with the data scanned from a 2D region, each point in space is transformed and projected in the direction of the scanning. An interpolation method may be used to carry each point not marked with a fiducial to coincide to a virtual reference point. The correction vectors may be generated relative to the stationary fiducial or relative to a selected reference image. The stationary fiducial is not necessary; for example, if the intrinsic registration of the 3D data set and the scanned data set are precise and reliable.

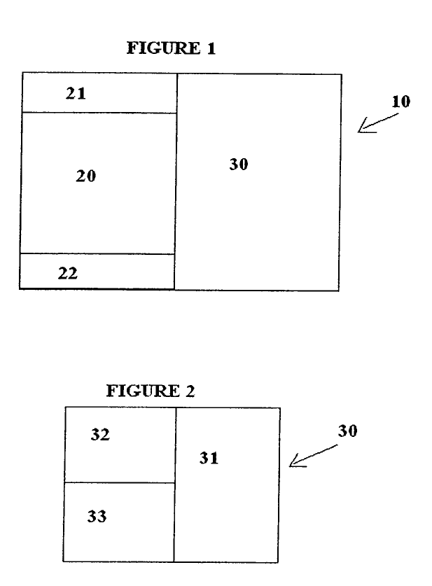

FIG. 1 shows one embodiment of an imaging system 10 for monitoring a medical process. The imaging system 10 may include an imaging device 20 and an image processing system 30. Additional, different, or fewer components may be provided.

The imaging system 10 is a medical workstation, an x-ray system, a biplane system, a computed tomography (CT) system, an angiography system, a magnetic resonance system, a fluoroscopy system, a C-arm system, a nuclear medicine system, a positron emission tomography (PET) system, a preclinical imaging system, a radiography system, a radiation oncology system, or other now known or latter developed imaging system. The imaging system 10 generates a dataset representing a region of the patient, and may generate an image of the region using the dataset.

The imaging system 10 may be used to plan and monitor a medical process, such as a needle procedure, surgery, or medical operation. The imaging system 10 may obtain data and generate images using the data. The imaging system 10 provides automated assistance to a physician for planning and monitoring a medical intervention. For planning, the imaging system 10 may be used to generate a three-dimensional (3D) representation of the medical intervention region prior to the medical process and plan the medical process using the 3D representation. For example, a needle path may be planned using the 3D representation. For monitoring, the imaging system may generate a two-dimensional (2D) image of the medical intervention region and register the 3D representation data and the 2D data. The imaging system 10 may fully or partially plan or monitor the medical intervention. For example, the imaging system 10 may provide automated assistance to a physician during a medical process by displaying a treatment device, such as a needle, in relation to the medical intervention area.

The imaging device 20 may include an imaging source 21 and an imaging detector 22. Additional, different, or fewer components may be provided. For example, the imaging device 20 may include two or more sources and/or two or more detectors, such as in biplane device.

The imaging device 20 may be a computed tomography (CT) device, a biplane device, a magnetic resonance device, an angiography device, a fluoroscopy device, a C-arm based X-ray system, other now known or later developed imaging devices, or any combination thereof. For example, the imaging device 20 may be a biplane device. The biplane device may operate as a CT device, such as a DynaCT device, to obtain a set of data representing a 2D region; for example, by rotation of a source and detector around the area to be imaged. The set of data representing a 2D region may be used to generate a 3D representation. The biplane device may also operate as a biplane fluoroscopy device with two detectors and two sources. The biplane fluoroscopy device may obtain data from at least two different angles at the same point in time. The data may be used to generate a 2D shadow projection. The projection may be a fluoroscopic projection.

The imaging source 21 and imaging detector 22 may be disposed opposite each other. For example, the imaging source 21 and imaging detector 22 may be disposed on diametrically opposite ends of a C-arm. In another example, the source 21 and detector 22 are connected inside a gantry. The region to be imaged (imaging region) is located between the source 21 and detector 22. The amount, shape, and/or angle of radiation may be adjusted to scan the region. All, a portion, or none of a patient may be disposed in the imaging region. For example, the medical intervention area in the patient, such as a human organ or a body part, may be disposed in the imaging region for generation of images of the intervention point.

In one embodiment, the imaging device 20 may include two or more sources and/or two or more detectors. For example, the imaging device 20 may be a biplane device having a first and a second C-arm, each C-arm has a source and detector disposed on diametrically opposite ends of the C-arms. The first and second C-arms may be moved relative to each other to obtain image data of the imaging region from two different directions at the same point in time.

The imaging source 21 may be a radiation source, such as an x-ray source. The imaging source 21 may emit radiation to the detector 22. The imaging detector 22 may be a radiation detector, such as a digital or film based x-ray detector. The imaging detector 22 may detect the radiation emitted from the imaging source 21. Image data is generated based on the amount or strength of radiation detected. For example, the imaging detector 22 detects the strength of the radiation received at the imaging detector 22 and generates image data based on the strength of the radiation. In an alternate embodiment, the imaging source 21 is a magnetic resonance source or other now known of later developed source.

The detector 22 detects data representing a two-dimensional (2D) region. The data represents a 2D imaging region from one direction. The data representing the 2D region may be used to generate a 2D image or combined with data from different directions to generate a three-dimensional (3D) representation. For example, the biplane system may be used to obtain data representing 2D regions from two different directions at the same time. The data may be used to generate a 2D shadow projection of the imaging region.

The data representing the 2D regions includes data relating to objects in the imaging region. The objects in the imaging region may include patient body parts, such as internal or external aspects of the human body, and/or fiducial markers. The image data may include data relating to additional, different, or fewer objects. For example, image data may include data relating to foreign objects, medical intervention tools, or other objects disposed in or on a patient.

A fiducial marker is an artificial maker, X-ray absorbent ball, or other known or later developed marker for marking a location during imaging. The fiducial marker has a high contrast ratio such that it appears in the image or images produced using the image data and may be easily identifiable from the patient body parts. For example, the contrast ratio of the fiducial may be such that a processor can identify the coordinates of the fiducial in a 2D or 3D coordinate system. The fiducial marker may be a metal alloy, a liquid-like substance, any now known or later developed marking material, or any combination thereof.

One or more fiducial markers may be mounted in stationary fashion or implanted in the human body. The stationary fiducial marker may be mounted on an object that is stationary relative to the patient or stationary relative to another object. For example, the fiducial marker may be disposed on a patient support, the floor, a base of the imaging system 10, or other stationary device. The implanted fiducial marker may be implanted in the patient using a needle, catheter, capsule, or any now known or later developed implanting device. A single fiducial marker may be implanted on a substantially rigid organ or body part, such as a kidney or bone. Alternatively, two or more fiducial markers may be implanted on (or in) an organ or body part, such as an abdomen, heart, bone, kidney, or lung.

A fiducial marker may be used in computerized image processing applications such as image fusion, image registration, or image overlay by providing an easy-to-track feature. The appearance of markers in images may act as a reference for image scaling, or may allow the image and physical object, or multiple independent images, to be correlated. By placing fiducial markers at known locations in the patient's body, the relative scale in the produced image may be determined by comparison of the locations of the markers in the image and subject. Images of the same subject produced with two different imaging systems might be correlated by placing a fiducial marker in the area imaged by both systems. In this case, a marker which is visible in the images produced by both imaging modalities must be used. For example, the 3D data set may be registered with the 2D projection. In another example, functional information from SPECT or positron emission tomography (PET) might be related to anatomical information provided by magnetic resonance imaging (MRI).

The coordinates of the fiducial(s) may be determined from at least two 2D images (e.g., from different directions) generated using the imaging system 10. For example, a physician may determine where to dispose the fiducials based on images obtained using the imaging device 20. The fiducials are disposed at coordinates or a location that may be imaged by the imaging device 20 during the medical procedure. The 2D images obtained using the imaging device 20 may represent the area that will be imaged during the medical procedure, so may be used as a guide as to the coordinates that the fiducials are disposed. For example, the imaging device 20 may be placed in medical procedure position (i.e., the position that it will be in during the medical procedure), the images obtained in this position may represent the boundaries for where to dispose the fiducials. Alternatively, the fiducials may be disposed in the patient and the imaging device 20 positioned so as to obtain at least two 2D images of the fiducial(s).

The imaging device 20 may be communicatively coupled to the image processing system 30. The imaging device 20 may be connected to the image processing system 30 by a communication line, cable, wireless device, communication circuit, or other communication device. For example, the imaging device 20 may communicate image data to the image processing system 30. In another example, the image processing system 30 may communicate an instruction, such as a position or angulation instruction, to the imaging device 20. All, some, or none of the image processing system 30 may be disposed in the imaging device 30. For example, the image processing system 30 may be disposed in the same or a different room as the imaging device 30, or in the same or different facilities.

As shown in FIG. 2, the image processing system 30 may include a processor 31, memory 32, and monitor 33. Additional, different, or fewer components may be provided. For example, the image processing system 30 may include an input device, such as a keyboard, mouse, compact disc drive or other now known or later developed input device.

The processor 31 is a general processor, digital signal processor, application specific integrated circuit, field programmable gate array, analog circuit, digital circuit, combinations thereof; or other now known or later developed processor. The processor 31 may be a single device or a combination of devices, such as associated with a network or distributed processing. Any of various processing strategies may be used, such as multi-processing, multi-tasking, parallel processing, or the like. The processor 31 is responsive to instructions stored as part of software, hardware, integrated circuits, firmware, micro-code or the like.

The processor 31 may generate an image, image representation, or image projection using the image data. The processor 31 processes image data received from the imaging device 20 and generates one or more fluoroscopic images, top-view images, in-plane images, orthogonal images, side-view images, 2D images, 3D representations, 2D projections, progression images, multi-planar reconstruction images, other now known or later developed images, or the combination thereof from the image data. For example, the processor 31 may generate a 3D representation from the image data obtained prior to the medical intervention and a 2D projection image from the image data obtained during the medical intervention.

The processor 31 may generate at least two 2D images from at least two different directions. The 2D images may used to plan the location or coordinates of the fiducials. The 2D images may represent the area to be imaged during a medical procedure. For example, the at least two 2D images may be obtained from a bi-plane system used to monitor the medical procedure.

The processor 31 may generate a 3D representation from a 3D image data set. The 3D data set may include data representing 2D planes from a plurality of different directions. The processor 31 combines the 2D data from a plurality of different directions to acquire the 3D representation.

The processor 31 may obtain the 3D data set from the imaging device 20, memory 32, or an input device. The 3D data set may be transferred to the processor 31. In another example, the processor 31 may retrieve the 3D image data set from the memory 32. The 3D image data set may be stored in memory 32 from a prior medical intervention or during a transfer of the patient. In another example, the processor 31 obtains the 3D image data from a network.

The 3D representation generated by the processor 31 may be a static representation. For example, the 2D image data used to generate the 3D representation may be obtained prior to the medical intervention and without patient body part movements. For example, the 2D image data may be obtained when the patient is holding their breath. In another example, the 2D image data may be obtained with an electrocardiogram [EKG or ECG] device for triggering acquisition of the data representing different planes each at a same point in the heart cycle.

The processor 31 may generate a 2D projection image. The 2D projection may be generated using a biplane system during the medical intervention. The 2D shadow projection may be a shadow projection and/or a fluoroscopic projection. Other images, representations, and projections may be generated based on the type of imaging device 20 being used to obtain the data.

The processor 31 may instruct the imaging device 20 to obtain image data at periodic times. For example, the processor 31 may instruct the imaging device 20, such as a CT, bi-plane device, or DynaCT device, to obtain 2D image data from two different directions and generate a 2D projection one (1) to sixty (60) times per second, and more preferably, ten (10), thirty (30), or sixty (60) times per second. The number of 3D representations generated during a defined time period or a length of the time, such as the time during a medical intervention, is not limited.

The processor 31 may display 2D images, 2D projections, and/or 3D representations on the display 33. The 2D images, 2D projections, and/or 3D representations may illustrate a portion of the patient's body with one or more fiducials. For example, the processor 31 may display a 2D shadow projection of the patient's body parts with fiducials on the display 33 and a 3D representation having the same fiducials on the display 33.

The processor 31 may perform image registration. The processor 31 may image register a 2D image and a 2D image; a 2D image and a 3D representation; a 2D projection and a 3D representation; or a 3D representation and a 3D representation. For example, the processor 31 may image register a 3D representation taken prior to the medical intervention and a 2D projection taken during the medical intervention. Image registration may occur during the medical intervention. Two different images, projections, and/or representations are registered and displayed on the monitor 33. Image registration may include mathematical process of geometrically overlaying a 2D image, 2D projection, or 3D representation with another 2D image, 2D projection, or 3D representation. For example, a 2D fluoroscopic projection may overlay a 3D representation obtained when the patient was motionless.

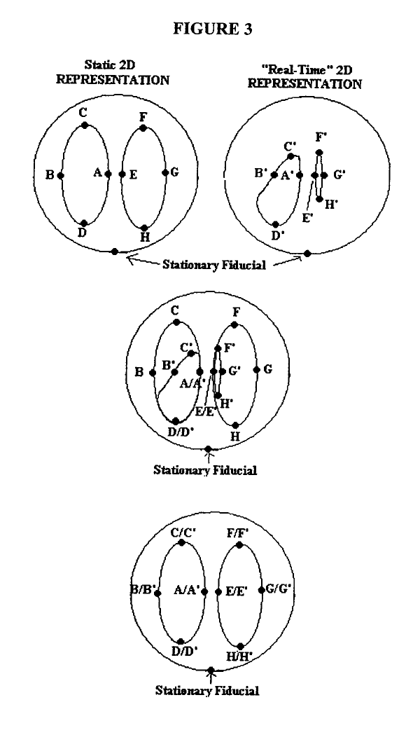

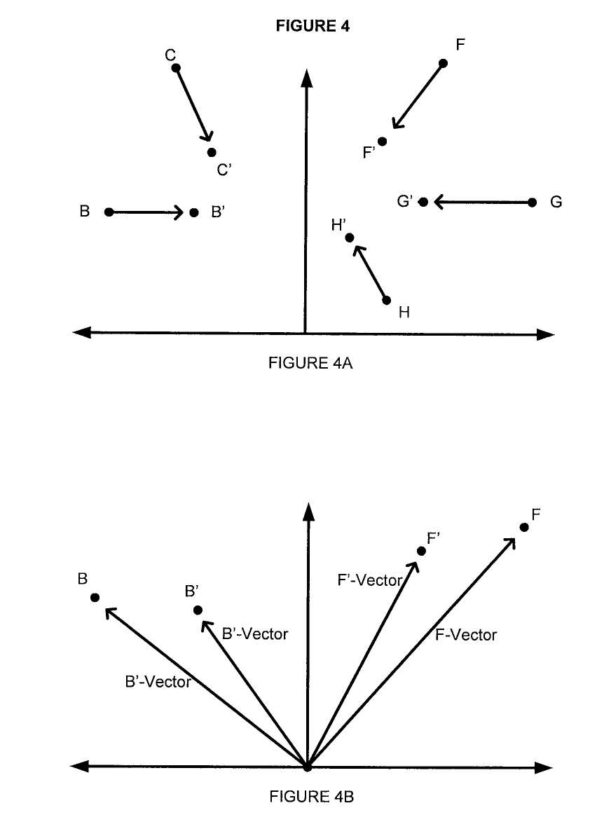

Image registration may include determining the fiducial coordinates in actual space and/or one of the coordinate systems for one of the data sets. The fiducial coordinates may be determined for one, two, or three dimensions. For example, the processor may determine the 2D coordinates for a 2D images and the 3D coordinates for a 3D representation. The fiducial coordinates in each of the 2D and 3D coordinate systems may be used to obtain correction vectors. The correction vectors indicate a change in location between the coordinate systems. The processor 31 may generate correction vectors for image registration. The correction vectors represent movement from a static position (e.g., prior to medical intervention) to a moved position (e.g., during medical intervention). The correction vectors may be generated relative to a selected reference image, as shown in FIG. 4A, or relative to the stationary fiducial, as shown in FIG. 4B. FIG. 4 illustrates a portion of the fiducials in FIG. 3.

Image registration may include identifying and marking one or more fiducials as reference locations. The fiducial may be a stationary fiducial, such that the reference location remains stationary from the time the 3D data set is obtained to the time the 2D projection data is obtained. The processor 31 may identify common reference locations for each 3D image representation or 2D projection. Alternatively, a physician or medical personnel may mark the stationary fiducials as reference locations using an input device. The location of the stationary fiducials may be identified and used for aligning, for example, a 3D representation with a 2D projection.

The processor 31 may overlay an image representation and an image projection using one or more stationary fiducials as reference points. Overlay may include geometrically aligning the stationary fiducials and/or the imaging regions. For 3D representations, this may include geometrically aligning the stationary fiducials and imaging regions in three dimensions (i.e., in the x, y, and z directions). For 2D representations, this may include aligning the stationary fiducials in two dimensions (i.e., in a 2D plane). The coordinates of one data set are translated and/or rotated to align the fiducials in each set.

FIG. 3 illustrates an example of registering a static 2D image of lungs, which was obtained prior to a medical intervention, and a “real time” 2D projection of the lungs, which was obtained during a medical intervention. In this example, prior to obtaining the static 2D image, the implanted fiducials (A-H) were implanted in the lungs and the stationary fiducial was disposed on the patient support. The location of fiducials may determine using images obtained from the imaging device used during the medical procedure, so that the imaging device is able to image the fiducials. Both the stationary and implanted fiducials are represented by data in the image data used for the 2D image. Prior to obtaining the image data for the 2D image, the patient inhaled and held their breath, so that the lungs were fully inflated. The medical intervention, such as a needle path or surgery path, was planned or mapped using the 2D image obtained from the image data.

During the medical intervention, image data is obtained for the “real time” 2D projection; for example, using a scanning device or a biplane imaging device. As shown in the “real time” 2D projection, at the time the image data for the 2D projection is taken, the lungs are only partially filled with air due to the patient's breathing. Accordingly, the patient's breathing caused the lungs to move, change shape, and/or change size.

Once the 2D image and 2D projection are obtained, the processor 31 provides automated assistance for image registering the 2D image and the 2D projection. For example, the processor 31 may geometrically align (overlay) the 2D image and the 2D projection using the stationary fiducial as the reference point. The stationary fiducial is identified in both images. For aligning (overlaying), the stationary fiducial of each image or data set are positioned at a same location. The middle illustration shown in FIG. 3 illustrates overlaying the representations and the movement of different portions of the patient's lungs. For example, the currently stationary portions of the pair of lungs, which are marked with the fiducial markers A, D, and E, did not change positions. Whereas other portions of the lungs, which are marked by fiducial markers B, C, F, G, and H, changed positions.

In an alternative embodiment, the imaging system 10 does not include a stationary fiducial disposed on a stationary device. For example, when the medical intervention point, such as a liver, is rigid and does not normally move, a stationary fiducials disposed outside the patient may not needed. Rather, implanted fiducials may be used to illustrate movement of the medical intervention point during the medical intervention.

The processor 31 may translate, deform, and/or interpolate different points of the images, projections, and or representations. Translation may include shifting one or more points from a first location to a second location. Translation may include a global or local shift. A global shift involves shifting each point in at least one of the images. For example, if fiducials A′-H′ moved positions, such as slightly to the left, the processor 31 may shift the entire image, projection, or representation to align the fiducials A′-H′ with the fiducials A-H, for example, by shifting the fiducials A′-H′ to the right by the offset amount. Alternatively, a local shift involves shifting a portion of the image For example, if the left lung (fiducials A′-D′) moved to the left, the processor 31 may shift the left lung (fiducials A′-D′) back to the right to align the fiducials A′-D′ with the fiducials A-D. In another example, as shown in the bottom illustration of FIG. 3, the position of the fiducials that changed positioned (C′, B′, F′, G′, H′) may be registered, such that the new positions correspond to the original positions (C, B, F, G, H). The “real time” projection now corresponds to the original image taken prior to the medical procedure and used to plan the medical procedure.

Deformation may include warping, zooming, matching, projecting, and changing the locations of the one or more fiducials. The locations of the one or more fiducials located in a second image, projection, or representation are deformed to coincide with the fiducials of a first image, projection, or representation. For example, as shown in FIG. 3, the point G′ would be matched with the point G. In another example, the point B′ may be changed to correspond with the point B.

The processor 31 may perform interpolation. Interpolation may include constructing new data points for each data point between and around the one or more fiducials. The processor 31 may interpolate points between and around the fiducials. Interpolation may include identifying a virtual marker for the first and second images, projections, and representations and interpolating each data point to coincide with the data points of the other image, projection, or representation. In another example, an area of the image representation may be shifted. The interpolation may be weighted, such as greater weighting a shift for locations closer to one fiducial by the translation associated with that fiducial. For locations halfway between two fiducials, equal weighting is used. Other interpolation weighting or extrapolation may be used.

The processor 31 may repeatedly register the 3D data set and the 2D projection without moving the imaging device 20. The processor 31 may register the data in real time. For example, the processor 31 may register the 3D data set and the 2D projection sixty (60) times per second. However, the number of times the processor 31 registers the 3D data set and the 2D projection is not limited. In another example, physician may obtain a real time display of the medical procedure without stopping the medical procedure to move the imaging device 20.

The processor 31 may communicate with the memory 32. The processor 31 and memory 32 may be connected by a cable, circuit, wireless-connection, or other communication coupling. Images, data, and other information may be communicated from the processor 31 to the memory 32 or vice-versa. For example, the processor 31 may communicate the generated images, image data, or other information to the memory 32. The processor 31 may retrieve information, images, image data, or other data from the memory 32. For example, the processor 31 may retrieve a medical intervention point stored in the memory 32. In another example, the processor 31 may retrieve patient data from the memory 32. The patient data may be used by the processor 31 to communicate instructions or requests to the imaging device 20. The patient data may include a patient's medical condition, size, positioning requirements, structural limitations, or other patient information. In another example, the processor 31 may retrieve structural limitation data stored in the memory 32.

The memory 32 is a computer readable storage media. The computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 32 may be a single device or a combination of devices. The memory 32 may be adjacent to, part of, networked with and/or remote from the processor 31.

The memory 32 may be a computer readable storage media having stored therein data representing instructions executable by the programmed processor 31 for monitoring a medical intervention. The memory 32 stores instructions for the processor 31. The processor 31 is programmed with and executes the instructions. The functions, acts, methods or tasks illustrated in the figures or described herein are performed by the programmed processor 31 executing the instructions stored in the memory 32. The functions, acts, methods or tasks are independent of the particular type of instructions set, storage media, processor or processing strategy and may be performed by software, hardware, integrated circuits, firm ware, micro-code and the like, operating alone or in combination. The instructions are for implementing the processes, techniques, methods, or acts described herein.

In one embodiment, a computer readable storage media stores data representing instructions executable by a programmed processor for monitoring a medical intervention. The instructions may include obtaining a 3D data set having data relating to one or more fiducials; obtaining a 2D projection data set having data relating to one or more fiducials; and placing the 2D projection data set in register with the 3D data set.

The memory 32 may store patient information. The patient information may be used to obtain one or more data set using the imaging device 20. For example, the information may relate to the size and condition of the patient. The size and condition information may be used to position the imaging device 20 to obtain a data set that corresponds to the correct area to be imaged.

The memory 32 may store image data sets. For example, the 3D data set obtained prior to the medical intervention may be stored in the memory 32. The processor 31 may retrieve the 3D data set for monitoring the medical intervention. In another example, an image data set may be stored for a subsequent medical intervention. The image data set may be used if the same area needs intervention at a later time.

The memory 32 may store a medical intervention plan. The medical intervention plan may be used for later medical interventions. For example, if a patient needs the same procedure performed, the medical intervention plan may be accessed and displayed as a reminder of how to perform the medical intervention. In another example, the memory 32 may be accessible via network, so that physicians in other facilities or at a later time may access the medical intervention plan as an example of how to perform the medical intervention.

The monitor 33 is a CRT, monitor, flat panel, a general display, LCD, projector, printer or other now known or later developed display device for outputting determined information. The monitor 33 may display one or more images. For example, the monitor 33 may display 2D images, projections, or representations. In another example, the monitor 33 may display 3D images, projections, or representations.

The monitor 33 may display the image registration. For example, the monitor 33 may display translation, deformation, and/or interpolation of an image, projection, or representation. One or more images may be displayed on the monitor 33. For example, a first image, projection, or representation may overlay a second image, projection, or representation on the monitor 33.

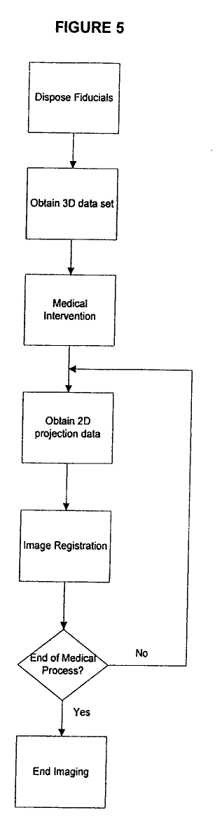

FIG. 5 shows a method for monitoring a medical intervention area during a medical procedure. The method may include disposing one or more fiducials 510; obtaining a 3D or 2D data set having data relating to the one or more fiducials 520; performing the medical intervention 530; obtaining a 2D projection or plane data set having data relating to the one or more fiducials 540; and placing the 3D data set in registration with the 2D projection data set 550. The method is implemented using the system 10 of FIG. 1 or a different system. Additional, different, or fewer acts than shown in FIG. 5 may be provided. For example, the method may include obtaining a 3D representation and a 2D projection relating to the same subject area and image registering the 2D projection and the 3D representation. The acts may be performed in the order shown or a different order. For example, act 540 may be performed before act 530. The acts may be performed automatically, manually, or the combination thereof.

In act 510, one or more fiducials are disposed in an imaging region. Disposing fiducials may include implanting, mounting, fixing, taping, attaching, or setting the fiducials. The fiducials may be disposed outside the patient, inside the patient, or the combination thereof The fiducials may be disposed outside the patient on a stationary device, such as a patient table or patient support. The fiducials may be disposed inside a patient on or in a body part, such as an organ. A first fiducial may be disposed outside the patient on a stationary device and a plurality of other fiducials may be disposed inside the patient. The placement of the fiducials is not limited. Any number of fiducials may be disposed at one or more locations that may be imaged, in any manner.

The one or more fiducials are disposed in an imaging region of the imaging device 20. The fiducials are disposed such that they are visible by the imaging device 20 for different scans. For example, the fiducials are disposed in two planes that may be imaged with the imaging device used during the medical procedure. For example, the location of the fiducials are determined from the 2D images obtained using a biplane imaging device. The fiducials may be scanned from the two planes. The planes may be orthogonal to one another or intersecting.

In act 520, an imaging device obtains a 3D or reference data set. The 3D data set includes data relating to the one or more fiducials. For example, the one or more fiducials are visible in an image generated from the 3D data set. Each of the fiducials disposed in the imaging region in act 510 may be visible in an image generated from the first 3D data set.

An imaging device may be used to obtain the 3D data set. Using an imaging device to obtain a 3D data set may include rotating a CT device around an area to be imaged to obtain 2D image data from a plurality of different directions. Alternatively, the 3D data set may be retrieved from a remote location or input device. For example, a stored 3D data set may be retrieved from a memory device, an input device, and/or from a remote location using a network.

In act 530, a medical intervention is performed. Performing the medical intervention may include planning, performing, and monitoring the medical intervention. Additional, different, or fewer acts may be provided. For example, medical intervention may include only performing and monitoring the medical intervention. The present embodiments are not limited to medical intervention. Other procedures may be performed, such as other medical procedures or product monitoring.

In act 540, an imaging device 20 obtains a 2D projection or other intervention data set. The 2D projection data set may include data relating to the one or more fiducials and may relate to the same area as the 3D data set. All or only a subset of the fiducials may be represented in the 2D projection data set. Obtaining the 2D projection data set may include using the imaging device 20 to obtain 2D image data from at least two different directions. The 2D image data from at least two different directions may be obtained at the same or substantially the same time. For example, a biplane device may obtain the 2D projection data set.

In act 550, the 3D data set and the 2D projection data set are registered with one another. Image registration may include overlaying, translation, deformation, and/or interpolation. Additional, different, or fewer acts may be provided.

Translation includes a local or global shift of data at a point marked by a fiducial. The data at a point marked by the fiducial is shifted to a point marked by a reference fiducial. For example, the premedical procedure data set may include data marked by one or more fiducials. The fiducials may be used as reference points. As the body parts in the imaging region move during the medical procedure, the data points in the medical procedure data set, which are marked by fiducials, are compared and shifted to the location of the corresponding reference fiducial.

The translation may be a local translation or a global translation. A local translation may include shifting one or more of the fiducials in the medical procedure data set to a location of a corresponding reference fiducial in the pre-medical procedure data set. A global translation may include shifting all of the fiducials in the medical procedure data set to locations that correspond to the reference fiducials in the pre-medical procedure data set.

Deformation may include warping, zooming, changing, aligning, or reforming. Deformation includes moving a fiducial in a medical process data set into a position that coincides with the fiducial location in a pre-medical process data set.

Interpolation includes moving a data point in a medical process data set into a position that coincides with the fiducial location in a pre-medical process data set. Interpolation may include moving data points between or around one or more fiducials. The interpolation may be weighted, such as greater weighting a shift for locations closer to one fiducial by the translation associated with the fiducial. For locations halfway between two fiducials, equal weighting is used. Other interpolation weighting or extrapolation may be used.

Image registration may include generating and displaying a 3D representation and a 2D projection. The 3D representation may be generated with a 3D data set and a 2D projection may be generated with 2D image data obtained from two different directions. The representation and projection may be displayed on a monitor. Alternatively, the 2D projection data may be registered with respect to the 3D data set and a projection may be generated based on the image registration. The image registration of the corrected 2D projection and 3D representation may be displayed on the monitor.

In act 570, if the medical intervention is still being performed, acts 540 through 550 are repeated until the end of the medical process. Acts 540 through 550 may be repeated at defined time periods. For example, the imaging system 10 may update the display 10, 30, or 60 times per second. The repetition may be non-periodically triggered, such as by a physician, in other embodiments.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A method for monitoring a medical procedure; the method comprising:
obtaining three-dimensional (3D) representation data using an imaging device;
obtaining two-dimensional (2D) projection data using the imaging device;
registering the 3D representation data and the 2D projection data using one or more fiducial as a reference point.

2. The method as claimed in claim 1, wherein obtaining 3D representation data may include obtaining 2D image data from two or more directions.

3. The method as claimed in claim 2, wherein obtaining 2D image data may include scanning.

4. The method as claimed in claim 1, wherein the imaging device includes a computed tomography device.

5. The method as claimed in claim 4, wherein the imaging device includes a DynaCT device.

6. The method as claimed in claim 1, wherein obtaining the 2D projection image includes obtaining image data from two different directions at a same time using the imaging device.

7. The method as claimed in claim 6, wherein the imaging device is a biplane imaging device.

8. The method as claimed in claim 1, wherein registering the 3D representation data and the 2D projection data includes image registration.

9. The method as claimed in claim 8, wherein image registration includes translation, deformation, interpolation, or any combination thereof.

10. The method as claimed in claim 1, the method comprising disposing the fiducial in an imaging region, such that the 3D representation data and 2D projection data have data relating to the fiducial.

11. The method as claimed in claim 10, wherein registering includes registering a data point marked with a first fiducial in the 2D projection data to coincide with a location of a corresponding fiducial in the 3D representation data.

12. The method as claimed in claim 10, wherein registering includes overlaying 2D projection data onto the 3D representation data.

12. The method as claimed in claim 1, the method comprising generating a 3D representation image from the 3D data set and a 2D projection image from the 2D projection data or the registered 2D projection data.

13. The method as claimed in claim 12, the method comprising displaying the 3D representation image and the 2D projection image.

14. An imaging system for planning and monitoring a medical procedure, the imaging system comprising:
an imaging device operative to obtain a 3D data set and a 2D projection data set;
one or more fiducial, the coordinates of the fiducial being planned using the imaging device; and
an image processing system communicatively coupled to the imaging device and operative to image register the 3D data set and the 2D projection data set from the imaging device, the image processing system being operable to use the one or more fiducial as reference points.

15. The imaging system as claimed in claim 14, wherein the imaging device includes a computed tomography (CT) device, a biplane device, or the combination thereof.

16. The imaging system as claimed in claim 15, wherein the computed tomography device includes a DynaCT device.

17. The imaging system as claimed in claim 15, wherein the imaging device is operative to use the CT device to obtain the 3D data set and the biplane device to obtain the 2D projection data set.

18. The imaging system as claimed in claim 17, wherein the biplane device is operative to obtain image data from two different directions at substantially the same time.

19. The imaging system as claimed in claim 17, wherein the CT device is operative to obtain image data by rotating around a patient area to be imaged.

20. The imaging system as claimed in claim 14, wherein image register includes translation, deformation, interpolation, or any combination thereof.

21. The imaging system as claimed in claim 14, wherein the 2D projection data includes fluoroscopic image data.

22. The imaging system as claimed in claim 14, wherein the one or more fiducials are disposed in the imaging region.

23. The imaging system as claimed in claim 22, wherein at least one of the one or more fiducials is disposed on a patient support that is stationary relative to a patient.

24. The imaging system as claimed in claim 23, wherein the at least one fiducial is used as a reference point for registering the 3D data set and the 2D projection data set from the imaging device.

25. Computer readable storage media having stored therein data representing instructions executable for monitoring a medical procedure, the instructions comprising:
obtaining a 3D data set and a 2D projection data set; and
image registering the 3D data set and the 2D projection data set.

26. The instructions as claimed in claim 25, wherein obtaining the 3D data set and the 2D projection data set include obtaining 2D image data from two or more directions.

27. The instructions as claimed in claim 25, wherein image registering includes translation, deformation, interpolation, or any combination thereof.

28. The instructions as claimed in claim 25, wherein image registering includes shifting or deforming a data point marked by a fiducial in the 2D projection data set to coincide with a data point marked by the fiducial in the 3D data set.

29. The method as claimed in claim 1, wherein the location of fiducials is determined using images obtained with the imaging device, the location being determined prior to obtaining the 3D representation data or 2D projection data.

30. A method for correcting displacements of a moving body area to be monitored during a medical procedure, the displacements being caused by movement of a patient; the method comprising:
implanting at least one stationary fiducial in the imaging area, the stationary fiducial remaining stationary during movement of the patient;
implanting at least one moving fiducial in the imaging area, the moving fiducial being disposed in the moving body area such that it may be displaced by movement of the patient,
obtaining a three-dimensional (3D) data set representing the imaging area and a two-dimensional (2D) projection data set representing the imaging area;
aligning or registering the 3D data set and the 2D projection data set using the at least one stationary fiducial; and
correcting displacements of the body area using the at least one moving fiducial as a reference point.

31. The method as claimed in claim 30, wherein registering includes shifting, transforming, warping, moving, altering, or repositioning a location of a moved artificial marker to the determined location of one or more artificial markers.

32. The method as claimed in claim 1, wherein registering includes using at least one stationary fiducial as a reference point for aligning or registering the 3D representation data and the 2D projection data with one another.

33. The method as claimed in claim 32, wherein registering includes correcting movements of a patient using at least one moving fiducial implanted in a moving portion of the patient as a reference point.

34. The method as claimed in claim 33, wherein movements of the patient may be caused by breathing or the patient's heartbeat.

35. The method as claimed in claim 34, wherein the moving portion of the patient is an organ.

36. The imaging system as claimed in claim 14, wherein the image processing system is operable to globally align the 3D data set and the 2D projection data set using a stationary fiducial and locally register the 3D data set and the 2D projection data set using at least one fiducial implanted in a moving body area of a patient.

37. The instructions as claimed in claim 25, wherein image registering the 3D data set and the 2D projection data set includes using at least one artificial marker as a reference point for aligning or registering the registering the 3D data set and the 2D projection data set with one another and using at least one fiducial being implanted in a moving body area as a reference point for correcting displacements of the moving body area.
